(19) United States
(12) Patent Application Publication
(10) Pub. No.: US 2024/0058258 A1
(43) Pub. Date: Feb. 22, 2024

(54) BIODEGRADABLE STRETCH FILM

(71) Applicants: Universite Paris-Saclay, ORSAY CEDEX (FR); Centre National de la Recherche Scientifique, PARIS CEDEX 16 (FR)

(72) Inventors: Raphaël Haumont, Linas (FR); Olesya Stukanova, Coulombs (FR)

(21) Appl. No.: 17/755,183

(22) PCT Filed: Oct. 23, 2020
(86) PCT No.: PCT/FR2020/051920

§ 371 Date: Sep. 16, 2022

(30) Foreign Application Priority Data
Oct. 23, 2019 (FR) FR1911831

(51) Int. Cl. A61K 8/9717; A61K 8/9794; A61Q 19/00; B65D 65/46; A61K 8/02; A45D 44/00; C08J 5/18

(52) U.S. Cl. CPC A61K 8/9717; A61K 8/9794; A61Q 19/007; B65D 65/466; A61K 8/0212; A45D 44/002; C08J 5/18; A61K 2800/882; A61K 2800/524; C08J 2305/00; C08J 2303/04

(57) ABSTRACT

A stretch film for use in the cosmetic or agri-food packaging sectors comprises konjac and carrageenan in a mass ratio of between 20/80 and 60/40. A stretch film according to the present disclosure has advantageous properties for use as a biodegradable stretch cling film and cosmetic mask.

### CROSS-REFERENCE TO RELATED APPLICATIONS

This application is a national phase entry under 35 U.S.C. § 371 of International Patent Application PCT/FR2020/051920, filed Oct. 23, 2020, designating the United States of America and published as International Patent Publication WO 2021/079076 A1 on Apr. 29, 2021, which claims the benefit under Article 8 of the Patent Cooperation Treaty to French Patent Application Serial No. FR1911831, filed Oct. 23, 2019.

### TECHNICAL FIELD

The present disclosure relates to the field of stretch films used in the cosmetic and agri-food packaging sectors. It relates more particularly to a stretch film comprising konjac and carrageenan in a mass ratio of between 20/80 and 60/40. A stretch film according to the present disclosure has advantageous properties for use as a cosmetic mask and biodegradable stretch cling film.

### BACKGROUND

Different types of stretch film are known from the state of the art. The article by Brenner T. et al. (Rheology and synergy of k-carrageenan/locust bean gum/konjac glucomannan gels; Carbohydrate Polymers 98 (2013) 754-760) describes various gels, some of which contain konjac and carrageenan, and compares their rheology. In the konjac and carrageenan gels studied, the carrageenan powder contains 14% KCl; these salts influence the properties of the gel.

Chinese Patent Application CN101298507 describes the preparation of a packaging film composed of material derived from biomass. The raw materials for making the film are KGM [(C20H32O16)n], carrageenan (C12H16SO10)n], glycerin (C3H8O3), sucrose fatty acid ester (C30O12H56) and water (H2O).

Chinese Patent Application CN108559276 describes a soluble edible film with added soy fiber. This film includes soy fiber as the main raw material, in which sodium carboxymethylcellulose, glycerin, konjac gum, and carrageenan are added to the solution. This film contains between 0.5 and 0.8% mass fraction of konjac gum and 0.5 and 0.6% mass fraction of carrageenan.

French Patent Application FR2963888A1 describes a particular cosmetic hydrogel comprising a gelling and thickening complex based on carrageenan, glucomannan and galactomannan. The hydrogel composition according to the invention comprises, optimally, between 0.2 and 2% carrageenan by hydrated weight of the hydrogel composition, between 0.2 and 2% glucomannan by hydrated weight of said composition, and between 0.2 and 2% galactomannan by hydrated weight of said composition.

Among the stretch films described above, some are used to make cosmetic masks. For example, International Patent Application Publication WO2006/053333 describes a cosmetic mask comprising an insoluble carrier and a gelled composition. The gelled composition is composed of two distinct gels, one comprising a polymeric, water-soluble gelling agent and the other comprising a reinforcing agent.

There are also 100% natural cosmetic masks based on bio-cellulose. However, cellulose is a highly modified molecule, to which chains are added to make it water soluble. Cellulose is therefore a processed product and not totally natural.

There is currently no 100% natural and biodegradable stretch film that has good elasticity and strength properties.

### BRIEF SUMMARY

It has been shown that the combination of konjac and carrageenan allows a gel to be obtained with very advantageous properties in terms of elasticity and strength, which can be stretched in the form of a film. These unique properties reflect the synergistic effect specific to the combination of konjac and carrageenan in proportions between 20/80 and 60/40.

Thus, the present disclosure relates to a composition of the biodegradable stretch film type comprising konjac and carrageenans in which:
the konjac/carrageenan mass ratio is between 20/80 and 60/40 and
the total mass percentage of konjac and carrageenan is between 0.5% and 2%.

The present disclosure also relates to the use of this composition for the preparation of a patch for topical use as well as kits for topical use comprising such a stretch film composition.

The present disclosure also relates to the use of this composition for the preparation of a biodegradable stretch cling film, as well as kits for preparing such a film.

Finally, the present disclosure relates to a method for preparing a stretch film as defined previously.

The stretch film according to the present disclosure has the advantage of being prepared solely from unprocessed biosourced ingredients. Since the carrageenans are derived from algae, the konjac from a tuber, and the products are not processed, the film offered is 100% natural and free of any toxic products. It is fully biodegradable and compostable.

This stretch film has unique properties that are particularly suitable for use as a cosmetic mask or patch. These properties are achieved only through the combination of konjac and carrageenans in a defined ratio. In other words, no other component is necessary to obtain these properties, unlike the generally known compositions of the prior art.

It can be used alone for its anti-drying properties and its cooling effect. Advantageously, a cosmetic composition is placed between the film and the skin.

The film constitutes a physical barrier with respect to the outside. Thus, the cosmetic composition deposited on the film is protected from oxidation during the contact time with the skin. Vitamins or other molecules sensitive to oxygen can act on the skin in an optimal way. The mask can be applied for several hours without drying out.

One of the problems that arises when a composition is applied to the skin is that it dries during the leave-in time, causing unpleasant feelings of tightness and difficulties during removal. When the cosmetic composition is protected by the stretch film serving as a mask support, it then does not dry out and the assembly remains comfortable. One advantage associated with this configuration is that the amount of cosmetic composition can be reduced compared to a conventional application, which is economical. This is all the more true since the film does not absorb the cosmetic composition, unlike certain fabric mask supports.

Another advantage of the film is that it adapts perfectly to the morphology of the surface on which it is applied. It can be the shapes of the face in the case of a cosmetic mask, but also any other part of the body on which one wishes to apply a patch: ankle, knee, hand, etc. This adaptability results from high elasticity combined with good breaking strength. The properties of this film give it a “second skin” effect. Thus, the film exerts a certain surface pressure on the skin that is conducive to the diffusion of the active principles through the superficial layers of the skin.

Surprisingly, the film produces a lingering cool effect. This effect is highly valued in many cosmetic and wellness applications. The cool effect is interesting for a cosmetic mask, in particular, for its decongestant effect (under the eyes, for example), but also for wellness applications on sunburn, to soothe the feeling of heavy legs, etc.

The properties of this film are also very interesting for making stretch cling films. Such films are intended to replace plastic films (polypropylene). Like the latter, the stretch films according to the present disclosure form a film used to preserve foods against oxidation and thus preserve their organoleptic properties.

It therefore involves a biopolymer film for biodegradable and compostable food use, in other words a plant-based cover, as well as kits for preparing such a film.

These stretch cling films can be applied against the edges of a container containing foodstuffs or directly in contact with foodstuffs. Being made of edible raw materials, they pose no health hazard even after prolonged direct contact with food.

The advantages described above for cosmetic applications can also be exploited in the agri-food industry. Indeed, owing to its great stretchability, the film adapts perfectly to the shape of the foods to be protected. It protects them from oxidation and dehydration for at least 24 hours, if not longer. It is easy to prepare and handle. Remember that it is compostable and biodegradable, as is food waste.

The stretch film according to the present disclosure can take different forms. A presentation in powder form allows one to make the film oneself, the preparation presents no difficulty. Indeed, it suffices to dilute a powder comprising a mixture of konjac and carrageenans in an aqueous solution. The stretch film can also directly take the form of a ready-to-use stretch film. Finally, the stretch film can be dehydrated and offered in the form of a sheet to be hydrated in an aqueous solution at the time of its use.

### BRIEF DESCRIPTION OF THE DRAWINGS

FIG. 1. Example of experimental penetrometry curves obtained on Kj-K gels.

FIG. 2. Evolution of the maximum penetration force as a function of the konjac fraction of the gel.

FIG. 3. Evolution of Young's modulus as a function of the konjac fraction of the gel.

FIG. 4. Evolution of the elongation at break as a function of the konjac fraction of the gel.

FIG. 5. Evolution of the maximum penetration force as a function of the konjac fraction of a gel containing 2% glycerin.

FIG. 6. Effect of protection against oxidation of a cling film according to the present invention deposited on the surface of the flesh of an avocado at t0, 1:20, 2:50, 11:00 and 16:00.

### DETAILED DESCRIPTION

A first object of the present disclosure relates to a composition of the biodegradable stretch film type comprising konjac and carrageenans in which:
the konjac/carrageenan mass ratio is between 20/80 and 60/40 and
the total mass percentage of konjac and carrageenan is between 0.5% and 2%.

In a particular embodiment, the mass ratio between konjac and carrageenans is between 25/75 and 50/50; this ratio makes it possible to reconcile good elasticity and good resistance to stretching.

From the point of view of the properties, the 35/65 ratio provides maximum synergy, but this ratio can be modulated according to the applications.

Advantageously, a ratio of between 30/70 and 55/45, or even between 35/65 and 55/45, allows suitable properties to be obtained for an application as stretch film.

For a mask application, a ratio between 40/60 and 45/55, and each of these two ratios, are particularly advantageous.

In a particular embodiment, a 60/40 ratio is possible when the solvent is distilled water.

Within the meaning of the present disclosure, “konjac” means a powder of Amorphophalus konjac, a vegetable in the form of a tuber grown in Southeast Asia. It can be a wholemeal flour obtained by grinding the tuber or glucomannan purified from konjac.

Advantageously, the carrageenans used are Kappa carrageenans or k-carrageenans.

The physical properties of the film are based solely on the synergistic effect observed owing to the mixture of konjac and carrageenan.

Thus, in a particular embodiment, the present disclosure relates to a composition of the biodegradable stretch film type containing konjac and carrageenans in which:
the konjac/carrageenan mass ratio is between 20/80 and 60/40 and
the total mass percentage of konjac and carrageenan is between 0.5% and 2%.

Other components can be added to the stretch film composition to give it additional properties without the elasticity and strength properties being impaired. Such components are, for example, maltodextrin, glycerin, a preservative, etc. In a particular embodiment, glycerin, an agent frequently used for its emollient, moisturizing and texturizing properties, in particular, in cosmetics and in pharmacy, is added to the composition.

A stretch film composition according to the present disclosure can be obtained by dissolving a powder comprising a mixture of konjac and carrageenan in the proportions defined above in an aqueous solution so that the total mass percentage of konjac and carrageenan is between 0.5 and 2%. Preferably, the total mass percentage of konjac and carrageenans is 1%.

Within the meaning of the present disclosure, “aqueous solution,” for example, means water, in particular, distilled water, a floral solution, an infusion of plants and/or fruits, or sea water.

In a particular embodiment of the present disclosure, the aqueous solution is seawater. This variant allows preparation of cosmetic masks and patches while benefiting from the richness in minerals, sought in, for example, thalassotherapy centers and by-products.

The stretch film composition can be in a dehydrated form.

In the context of the present disclosure, “stretch film composition in dehydrated form” means a dry composition obtained by dehydration of a film having elasticity and strength properties as defined above.

A stretch film composition in dehydrated form can more particularly be obtained by subjecting a stretch film comprising carrageenans and konjac as defined above to a dehydration method. Different dehydration methods are well known to those skilled in the art, namely, for example, drying at room temperature, heating, etc.

Such a dehydrated composition is intended to be rehydrated before use, for example, by soaking in an aqueous solution.

A second object of the present disclosure relates to the use of a composition as defined above as a patch for topical use.

The mechanical properties of this gel are quite suitable for application to the skin.

Used alone in the form of a patch or mask, this film provides a feeling of coolness and hydration.

Used in combination with a cosmetic or wellness composition, it allows a “second skin”-type application while preventing the composition from drying out and oxidizing. The feeling of tightness is avoided, and the active ingredients are preserved and kept in contact with the skin. In this case, the film can be considered as a support for topical application of a cosmetic or wellness composition.

A third object of the present disclosure relates to a kit for topical use comprising a composition as defined previously and at least one ingredient for cosmetic use.

In a particular embodiment, the present disclosure relates to a ready-to-use kit comprising a stretch film, in hydrated or dehydrated form, and at least one ingredient for cosmetic or wellness use.

The ingredient for cosmetic or wellness use can be one or more ingredients to be diluted in a solution or be a cosmetic composition. The ingredient or the cosmetic or wellness composition can be chosen from those known to those skilled in the art for their value in cosmetics or wellness, such as a moisturizing agent, a vitamin, a vegetable oil, an essential oil, etc., or any combination thereof.

In practice, the user must first prepare the film and then spread the active principles on the surface thereof, for example, using a brush.

A “wellness composition” can contain, for example, essential oils with soothing or stimulating properties, Bach flowers or any other ingredient or composition that can act on physical or mental well-being.

When the kit contains a mask or a patch in dehydrated form, the kit can also comprise an aqueous solution to rehydrate the stretch film.

A fourth object of the present disclosure relates to the use of a composition as defined above as a biodegradable stretch cling film.

This involves using a composition according to the present disclosure to prepare a stretch cling film.

A fifth object of the present disclosure relates to a kit for the preparation of a biodegradable stretch cling film.

In a particular embodiment, the ready-to-use kit comprises a stretch film, in hydrated or dehydrated form. When the kit contains a film in dehydrated form, it may also comprise an aqueous solution for rehydrating the stretch film and possibly a mold for casting the film.

A sixth object of the present disclosure relates to a method for preparing a composition as defined above, this method involves diluting a powder comprising at least a mixture of konjac and carrageenan in an aqueous solution, the konjac/carrageenan mass ratio being between 20/80 and 60/40 and the total mass percentage of konjac and carrageenans being between 0.5% and 2%.

The present disclosure will be better understood on reading the examples that follow, which are provided by way of illustration and should in no case be considered as limiting the scope of the present disclosure.

### EXAMPLES

### Example 1: Preparation of Konjac/Carrageenan Gels

The gels were made with distilled water or seawater (salinity of 30 g/L).

Various mixtures of konjac gum (Kj) and carrageenan kappa (κ) were tested, working at 1% in total mass of texturizers. The ratio x=Kj/(Kj+K), also called “konjac fraction” hereinafter, varies from 0 to 1.

The texturizers are dispersed in the water. The mixture is brought to 90° C. for 30 s with stirring. The gel is poured and then cooled at 8° C. for 12 hours. Physical measurements are performed at room temperature.

### Example 2: Characterization Methods for Gels

In order to characterize the gels, texturometry measurements were carried out.

The physical measurements are carried out on an Ametek TA texturometer. The probe is a cylinder 12.6 mm in diameter. The measurements are done in compression, up to 60% of the initial height of the sample.

The modulus of elasticity (Young's modulus), the maximum penetration force as well as the elongation at break (if observed) are measured. Young's modulus and the maximum force are expressed in gram-force (gf). The elongation at break is expressed in % compared to the initial length.

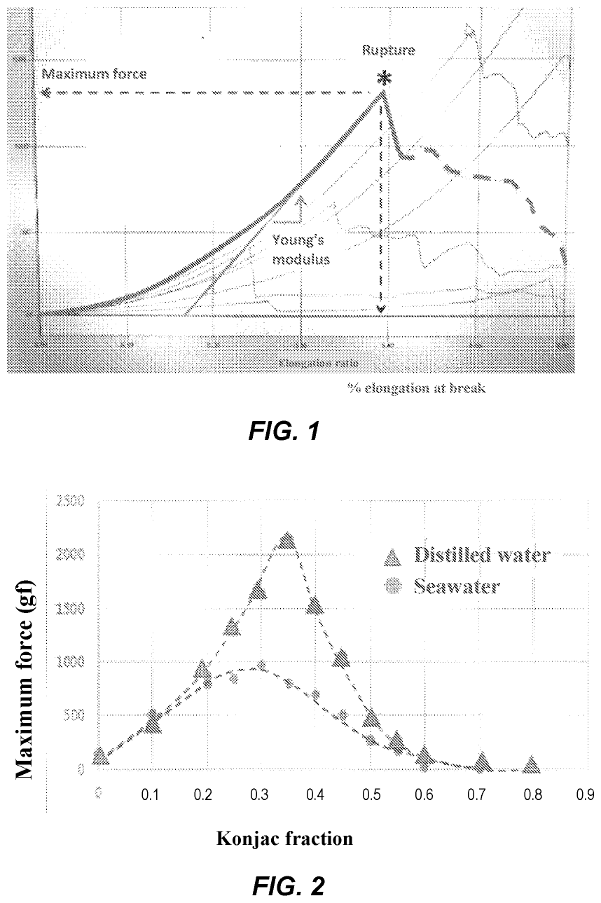

An example of the curve obtained is shown in FIG. 1. The physical quantities extracted from the curves are indicated. Remark: The dotted lines indicate parts of the curves that cannot be used (artifacts of the probe following the rupture of the sample).

### Example 3: Mechanical Properties of Konjac/Carrageenan Gel

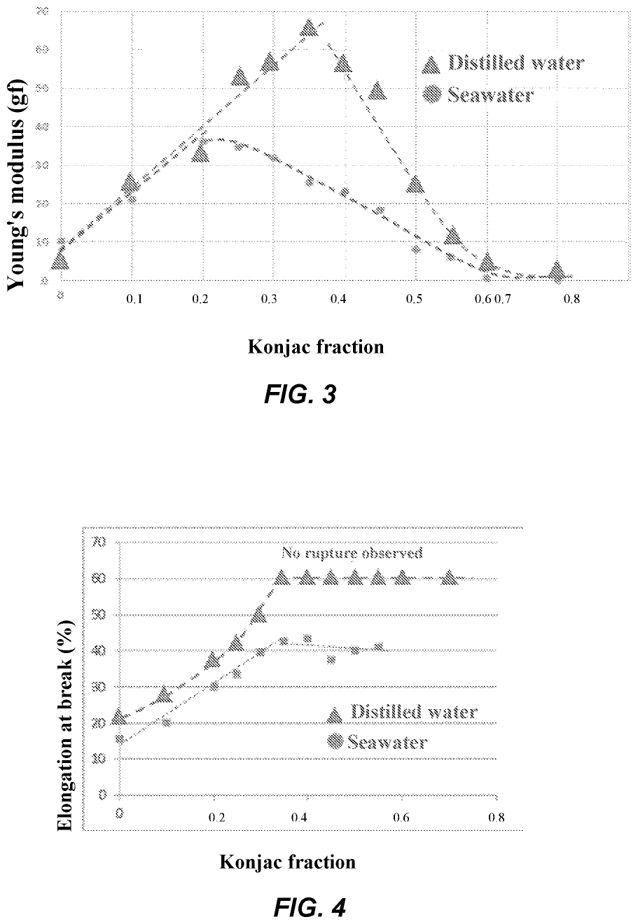

FIGS. 2 and 3 show the maximum force and the Young's modulus of the different gels as a function of the konjac fraction.

Synergy of the konjac-carrageenan mixture

Adding konjac to a carrageenan gel greatly increases the mechanical properties. Indeed, it is observed that the Young's modulus doubles with an addition of only 10% konjac gum. This underlines the synergistic nature of the konjac-carrageenan mixture.

The synergy is also noticeable by the non-linearity of the measurements. Young's modulus and the elongation force increase when x<0.35, then decrease beyond this critical value. There is no proportionality between x and the physical quantities over the entire range 0<x<1.

There is an optimal value estimated at x ˜0.35 (distilled water) and x ˜0.25-0.3 (seawater) for which the physical quantities pass through maxima. This means that the synergistic effect is the greatest and the gels are the most elastic and resistant around this critical ratio.

The konjac-carrageenan synergy is very resistant and particularly remarkable: a carrageenan gel containing 35% konjac (x=0.35) is more than 10 times more resistant than a pure carrageenan gel (Young's modulus goes from 5.7 to 65.1 with the addition of konjac). The force required to deform the gel is multiplied by more than 25 at this concentration range (90 vs 2400).

Salt (NaCl), present in seawater, seems to decrease the physical properties of the gels, although the gels nevertheless show remarkable properties. The maximum synergy is slightly lowered toward low concentrations of konjac.

Elongation at Break of the Gels

FIG. 4 shows the elongation at break of the gels.

While a pure carrageenan gel is “brittle” (elongation at break observed at approximately 20%) and not very resistant (low Young's modulus and low breaking force), the addition of konjac greatly increases this elongation: beyond the threshold value x ˜0.35, the gel does not break (in the range of measurements carried out) and retains a very strong elasticity. This absence of rupture can be associated with “stretchability.” This underlines a very strong cohesion of the konjac-carrageenan chains under mechanical stress. The value x ˜0.35, again noted on this graph, once again demonstrates a maximum of synergy for this particular mixture.

Properties of Gels Comprising Glycerin

Glycerin was added to the composition at the level of 2%.

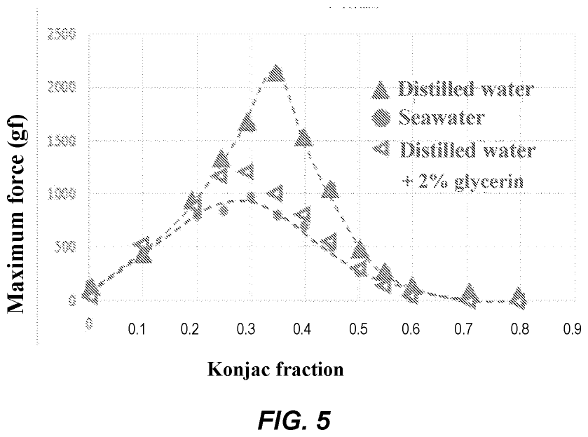

The properties of this gel have been studied. The results are shown in FIG. 5.

The optimal value is estimated at x=0.30, that is to say, at a value similar to that estimated for the gels prepared without glycerin. This result attests to the fact that the addition of an additional ingredient has little influence on the properties of the gels and that the synergistic effect is therefore very specific to the konjac/carrageenan combination.

### Conclusion

When x evolves from 0 to 1, gels are obtained with very different properties. These properties are easily modulated according to the value of x. Even if seawater or the addition of an ingredient such as glycerin alters these properties and shifts the resistance maxima, it is very easy to adapt the desired properties with x depending on the application environment:
Low values of x: brittle, fragile, weak gel. This is characteristic of a carrageenan gel (but also gelatin, agar-agar, etc. gel).
For 0.2<x<0.6, the gels are very elastic, resistant and stretchable. A maximum of properties is observed around x ˜0.35 in distilled water and x ˜0.25-0.3 in seawater. The konjac-carrageenan chemical synergy explains these non-linear properties and the high values of measured physical quantities.
High values of x: the gel is “sticky,” or even no longer forms beyond x ˜0.7-0.8. A very viscous solution is obtained. This is characteristic of a pure solution of konjac gum (non-gelling thickener, just like, but on a lesser scale, locust bean gum, guar gum, gum arabic, etc.).

### Example 4: Thermal Properties of the Gels

In order to test their resistance and their reversibility to heat, konjac-carrageenan gels (with x=0.3 and x=0.5) were heated at 121° C., 110 kPa for 35 min. The properties of the gels before heating and after heating/cooling were compared.

This experiment showed that the elasticity and resistance properties are found again after cooling. It is therefore possible to sterilize the gels before packaging in order to allow them to be stored in a protected atmosphere.

### Example 5: Cool Effect of the Gels

It has been observed that deposited on the skin, the films described above provide a cool effect, which persists for several tens of minutes. This cool/cold sensation is independent of the temperature of the film, since the film is initially at room temperature and is at the temperature of the skin at the end of the experiment. The cool/cold sensation is not related to excess moisture or water exuding from the gel by syneresis (water vaporizing on the surface of the skin as experienced when a body exits water, for example). By comparison, pure gels of carrageenan, agar-agar, or even gelatin gels have a much less marked and more ephemeral cooling effect.

### Example 6: Protective Effect Against Oxidation

An avocado was cut in two. On one of the halves, a film of carrageenan/konjac (x=0.4) 1 mm thick was deposited, in direct contact with the flesh of the vegetable.

The oxidation reaction (polyphenol oxidases, which react with oxygen to lead to brown pigments) was observed over time.

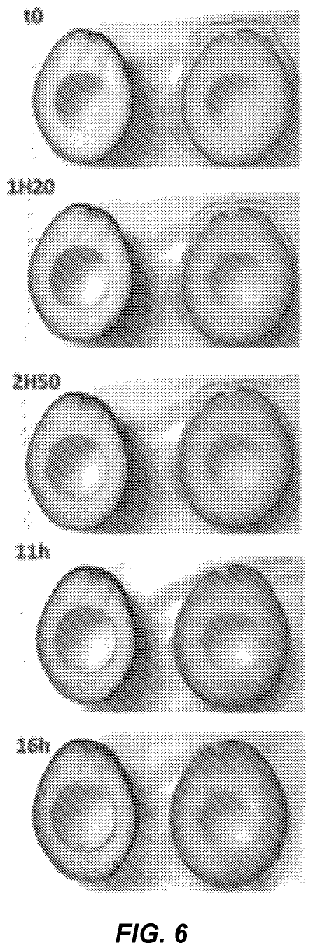

The result is shown in FIG. 6.

It is found that the flesh is preserved from oxidation reactions for at least 16 hours. The oxidation reaction is therefore greatly slowed down in the presence of the film, which shows that the gel prevents the diffusion of oxygen. This is of major interest both in food and in cosmetics (where many organic active principles are unstable in air).

Moreover, as observed in the photos of FIG. 6, dehydration is considerably slowed down on the vegetable covered with the film. The initial volume (½ avocado) is preserved throughout the duration of the experiment (16 hours) for the part covered with the plant-based film, whereas for the part left in the open air, a gradual reduction in volume and a “shriveling” of the flesh is noticed. The water-rich film prevents water migration from the vegetable.

This experiment, carried out on an avocado, can be generalized to all foods (fruits, vegetables, fish, meat, etc.).

Tests have also been carried out using the film as a cover (similar to current plastic-based stretch films): the film is not in contact with the food, but is stretched over the surface of a container containing the food. The film dries over time in the refrigerator, but the food remains preserved from oxidation and dehydration, for several dozen hours. Once removed, the film is compostable.

In conclusion, this experiment shows that the film allows the freshness of food to be preserved, in particular, by avoiding surface oxidation and dehydration. Thus, the film plays the role of a physical barrier making it possible to isolate a food here, but in a cosmetic application, the surface of the skin, from the drying and oxidizing effects of the surrounding air.

1. A biodegradable stretch film composition, comprising: konjac and carrageenans, and
wherein the konjac/carrageenan mass ratio is between 20/80 and 60/40; and
wherein the total mass percentage of konjac and carrageenan is between 0.5% and 2%.

2. The composition of claim 1, further comprising a component selected from maltodextrin, glycerin or a preservative.

3. The composition of claim 1, wherein the composition is in dehydrated form.

4. A method, comprising:
providing a biodegradable stretch film composition comprising konjac and carrageenans,
wherein the konjac/carrageenan mass ratio is between 20/80 and 60/40, and wherein the
total mass percentage of konjac and carrageenan is between 0.5% and 2%; and applying the film to the skin of human subject.

5. A method, comprising:
providing a biodegradable stretch film composition comprising konjac and carrageenans,
wherein the konjac/carrageenan mass ratio is between 20/80 and 60/40, and wherein the
total mass percentage of konjac and carrageenan is between 0.5% and 2%; and applying the film to a food product.

6. A kit for topical use, comprising:
a biodegradable stretch film composition comprising konjac and carrageenans, wherein the
konjac/carrageenan mass ratio is between 20/80 and 60/40, and wherein the total mass
percentage of konjac and carrageenan is between 0.5% and 2%; and
at least one ingredient for cosmetic use.

7. The kit of claim 6, wherein the at least one ingredient is chosen from among a moisturizing agent, a vitamin, a vegetable oil, and an essential oil.

8. The kit of claim 6, wherein the composition further comprises a component selected from maltodextrin, glycerin or a preservative.

9. The kit of claim 6, wherein the composition is in dehydrated form, and wherein the kit further comprises an aqueous solution for rehydrating the composition.

10. A method of forming a composition for use in forming a biodegradable stretch film, the method comprising:
diluting a powder comprising at least one mixture of konjac and carrageenan in an aqueous solution, the konjac/carrageenan mass ratio being between 20/80 and 60/40 and the total mass percentage of konjac and carrageenans being between 0.5% and 2%.

11. The composition of claim 2, wherein the composition is in dehydrated form.

12. The kit of claim 7, wherein the composition is in dehydrated form, and wherein the kit further comprises an aqueous solution for rehydrating the composition.
